(19) United States
(12) Patent Application Publication
(10) Pub. No.: US 2009/0012167 A1
(43) Pub. Date: Jan. 8, 2009

(54) Omega-3 Fatty Acids and Dyslipidemic Agent for Lipid Therapy

(75) Inventors: Roelof M. L. RONGEN, Caliton, NJ (US); George BOBOTAS, Tarpon Springs, FL (US); Egil BODD, Oslo (NO); Hogne VIK, Eiksmarka (NO)

Correspondence Address: ARENT FOX LLP, 1050 CONNECTICUT AVENUE, N.W., SUITE 400, WASHINGTON, DC, 20036, US

(73) Assignees: RELIANT PHARMACEUTICALS, INC., Liberty Corner, NJ (US); PRONOVA BIOCARE AS, Lysaker (NO)

(21) Appl. No.: 12/210,872

(22) Filed: Sep. 15, 2008

Related U.S. Application Data
(62) Division of application No. 11/284,095, filed on Nov. 22, 2005, now abandoned.
(60) Provisional application No. 60/633,125, filed on Dec. 6, 2004.
(60) Provisional application No. 60/699,866, filed on Jul. 18, 2005.
(60) Provisional application No. 60/659,099, filed on Mar. 8, 2005.

(51) Int. Cl. A61K 31/202; A61P 7/00

(52) U.S. Cl. 514/560

(57) ABSTRACT

A method and composition for blood lipid therapy by administering to the subject an effective amount of a dyslipidemic agent and omega-3 fatty acids. The method utilizes a single administration or a unit dosage of a combination of dyslipidemic agent and omega-3 fatty acids for the treatment of patients with hypertriglyceridemia, hypercholesterolemia, mixed dyslipidemia, coronary heart disease (CHD), vascular disease, artherosclerotic disease and related conditions, and the prevention or reduction of cardiovascular and vascular events.

The present application claims priority from provisional patent application Ser. No. 60/633,125, filed Dec. 6, 2004, Ser. No. 60/659,099, filed Mar. 8, 2005, and Ser. No. 60/699,866, filed Jul. 18, 2005. The disclosure of the provisional applications is hereby incorporated by reference.

### FIELD OF THE INVENTION

The present invention relates to a method utilizing a single administration or a unit dosage of a combination of a dyslipidemic agent and omega-3 fatty acids for the treatment of patients with hypertriglyceridemia, coronary heart disease (CHD), vascular disease, artherosclerotic disease and related conditions, and the prevention or reduction of cardiovascular and vascular events.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C and VLDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C. In addition, researchers have found that non-HDL cholesterol is an important indicator of hypertriglyceridemia, vascular disease, artherosclerotic disease and related conditions. In fact, recently non-HDL cholesterol reduction has been specified as a treatment objective in NCEP ATP III.

Agents, such as dyslipidemic agents and omega-3 fatty acids, have been used to treat post-myocardial infarction (MI) and adult endogenous hyperlipidemias of hypercholesterolemias and of hypertriglyceridemias, which are generally categorized as “cardiovascular events”.

Dyslipidemic agents commonly include HMG CoA inhibitors (statins), cholesterol absorption inhibitors, niacin and derivatives such as nicotinamide, fibrates, bile acid sequestrants, MTP inhibitors, LXR agonists and/or antagonists and PPAR agonists and/or antagonists.

Statins, which are 3-hydroxy-3-methyl glutaryl coenzyme A (HMG-CoA) reductase inhibitors, have been used to treat hyperlipidemia and arthrosclerosis, for example. Typically, statin monotherapy has been used to treat cholesterol levels, particularly when a patient is not at an acceptable LDL-C level. Statins inhibit the enzyme HMG-CoA reductase, which controls the rate of cholesterol production in the body. Statins lower cholesterol by slowing down the production of cholesterol and by increasing the liver's ability to remove the LDL-cholesterol already in the blood. Accordingly, the major effect of the statins is to lower LDL-cholesterol levels. Statins have been shown to decrease CHD risk by about one-third. However, statins only appear to have a modest effect on the TG-HDL axis.

Cholesterol absorption inhibitors, such as ezetimibe and MD-0727, are a class of lipid-lowering compounds that selectively inhibit the intestinal absorption of cholesterol. Ezetimibe acts on brush border of the small intestine and decreases biliary and dietary cholesterol from the small intestine uptake into the enterocytes.

Cholesteryl ester transfer protein (CETP) inhibitors, such as torcetrapib, inhibit the CETP molecule which, among other things, moves cholesterol from the HDL form to the LDL form. Inhibiting this molecule is, therefore, a promising approach to increasing HDL cholesterol levels.

Niacin (nicotinic acid or 3-pyridinecarboxylic acid) has previously been used to treat hyperlipidemia and atherosclerosis. Niacin is known to reduce total cholesterol, LDL-C and triglycerides and increase HDL-C. Niacin therapy is also known to decrease serum levels of apolipoprotein B (Apo B), the major protein component of VLDL-C and LDL-C fractions. However, the magnitude of the individual lipid and lipoprotein response from niacin therapy may be influenced by the severity and type of underlying lipid abnormality.

Fibrates such as fenofibrate, bezafibrate, clofibrate and gemfibrozil, are PPAR-alpha agonists and are used in patients to decrease lipoproteins rich in triglycerides, to increase HDL and to decrease atherogenic-dense LDL. Fibrates are typically orally administered to such patients.

Fenofibrate or 2-[4-(4-chlorobenzoyl)phenoxy]-2-methyl-propanoic acid, 1-methylethyl ester, which belongs to the fibrate family, has been known for many years as a medicinal active principle because of its efficacy in lowering blood triglyceride and cholesterol levels. Fenofibrate is an active principle which is very poorly soluble in water and the absorption of fenofibrate in the digestive tract is limited. A treatment of 40 to 300 mg of fenofibrate per day enables a 20 to 25% reduction of cholesterolemia and a 40 to 50% reduction of triglyceridemia to be obtained.

Bile acid sequestrants, such as cholestyramine, colestipol and colesevelam, are a class of drugs that binds bile acids, prevents their reabsorption from the digestive system, and reduces cholesterol levels. The usual effect of bile acid sequestrants is to lower LDL-cholesterol by about 10 to 20 percent. Small doses of sequestrants can produce useful reductions in LDL-cholesterol.

MTP inhibitors, such as implitapide, are known to inhibit the secretion of cholesterol and triglyceride.

Liver X receptors (LXRs) are “cholesterol sensors” that regulate the expression of genes involved in lipid metabolism in response to specific oxysterol ligands (Repa et al., Annu. Rev. Cell Dev. Biol. 16: 459-481 (2000)). LXR agonists and antagonists are potential therapeutic agents for dyslipidemia and atherosclerosis.

PPAR-gamma agonists, such as the thiazolidinediones pioglitazone and rosiglitazone, have been shown to improve surrogate markers of cardiovascular risk and atherosclerosis. For example, thiazolidinediones decrease C-reactive protein and carotid intima-media thickness. Non-thiazolidinediones, such as tesaglitazar, naviglitizar and muraglitazar, are dual alpha/gamma PPAR agonists. These compounds are used for lowering glucose, insulin, triglycerides and free fatty acids.

Partial PPAR-gamma agonist/antagonists, such as metaglidasen, are used for the treatment of type II diabetes.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One such form of omega-3 fatty acid is a concentrate of omega-3, long chain, polyunsaturated fatty acids from fish oil containing DHA and EPA and is sold under the trademark Omacor®. Such a form of omega-3 fatty acid is described, for example, in U.S. Pat. Nos. 5,502,077, 5,656,667 and 5,698,594, each incorporated herein by reference.

Patients with mixed dyslipidemia or hypercholesteremia often present with blood levels of LDL cholesterol greater than 190 mg/dl and triglyceride levels of 200 mg/dl or higher. The use of diet and single-drug therapy does not always decrease LDL cholesterol and triglycerides adequately enough to reach targeted values in patients with mixed dyslipidemia or hypercholesterolemia with or without a concomitant increase in triglycerides. In these patients, a complementary combination therapy of a dyslipidemic agent and omega-3 fatty acids may be desirable.

Studies have examined the effects of fish oil and statin therapy. One study found that fish oil and lovastatin increases plasma LDL cholesterol and VLDL cholesterol. Saify et al., Pakistan J. of Pharm. Sci. (2003) 16 (2): 1-8. Nakamura et al. investigated the effects of purified EPA and statins on patients with hyperlipidemia. Patients having baseline triglyceride levels of 2.07 mmol/l (about 182 mg/dl) and already treated with 5-20 mg/day pravastatin or 5 mg/day simvastatin were additionally treated for 3 months with 900 or 1800 mg/day purified (>90%) EPA ethyl ester. It was reported that combination treatment significantly reduced triglyceride levels, and significantly increased HDL-C levels, as compared to baseline monotherapy. LDL-C levels were not reported. Nakamura et al., Int. J. Clin. Lab Res. 29:22-25 (1999).

Davidson et al. investigated the effects of marine oil and simvastatin in patients with combined hyperlipidemia. Patients having baseline triglyceride levels of 274.7 mg/dl to 336.8 mg/dl were treated for 12 weeks with 10 mg/day simvastatin and placebo, 7.2 g/day marine oil (SuperEPA® 1200) and placebo, or a combination of simvastatin and SuperEPA®. The content of omega-3 fatty acids in 7.2 g of marine oil used in the study was 3.6 g, with an EPA/DHA ratio of 1.5. Combination treatment was shown to significantly increase HDL-C levels, as compared to marine oil alone. In addition, triglyceride and non-HDL-C levels were significantly reduced with combination treatment. However, non-HDL-C levels were reported to be reduced less with combination treatment than with simvastatin alone. Davidson et al., Am J Cardiol (1997) 80: 797-798.

Hong et al. investigated the effects of fish oil and simvastatin in patients with coronary heart disease and mixed dyslipidemia. Patients having baseline triglyceride levels of 292.8 mg/dl or 269.5 mg/dl were initially treated with 10-20 mg/day simvastatin for 6-12 weeks. Thereafter the patients were treated with simvastatin and placebo or simvastatin and 3 g/day fish oil (Meilekang™). Combined treatment significantly reduced triglyceride levels, as compared to baseline and placebo. In addition, combined treatment numerically increased HDL-C levels, and numerically reduced LDL-C levels, as compared to baseline. However, the changes in HDL-C levels and LDL-C levels were not statistically significant. Hong et al., Chin. Med. Sci. J. 19:145-49 (2004).

Contacos et al. investigated the effects of fish oil and pravastatin on patients with mixed hyperlipidemia. Patients having baseline triglyceride levels of 4.6 to 5.5 mmol/l (404 to 483 mg/dl) were initially treated for 6 weeks with 40 mg/day pravastatin, 6 g/day fish oil (Himega™, containing 3 g of omega-3 fatty acids, with an EPA/DHA ratio of 2:1), or placebo. Thereafter, all patients were treated with pravastatin and fish oil for an additional 12 weeks. Initial treatment with pravastatin significantly reduced LDL-C levels. Combined treatment of pravastatin and fish oil also significantly reduced triglyceride and LDL-C levels. However, the addition of fish oil to pravastatin monotherapy resulted in only a numerical increase in LDL-C levels, which was not statistically significant. Treatment with fish oil alone significantly reduced triglyceride levels, but increased LDL-C levels. Combined treatment for this group significantly reduced LDL-C levels, as compared to fish oil alone (but not as compared to baseline). Contacos et al., Arterioscl. Thromb. 13:1755-62 (1993).

Singer investigated the effects of fish oil and fluvastatin on patients with combined hyperlipidemia. Patients having baseline triglyceride levels of 258 mg/dl were initially treated for two months with 40 mg/day fluvastatin, and thereafter were additionally treated for two months with 3 g/day fish oil (18% EPA and 12% DHA). Thereafter, the patients remained on fluvastatin therapy alone for a final two months. Fluvastatin monotherapy was shown to significantly reduce triglyceride and LDL-C levels, and significantly increase HDL-C levels. Combination treatment significantly reduced triglyceride and LDL-C levels and resulted in an additional numerical reduction of triglyceride and LDL-C levels, as compared to fluvastatin alone. Combination treatment numerically increased HDL-C levels, as compared to monotherapy, although the increase in HDL-C levels with combined treatment was not statistically significant. Singer, Prost. Leukotr. Ess. Fatty Acids 72:379-80 (2005).

Liu et al. investigated the effects of fish oil and simvastatin in patients with hyperlipidemia. Patients having baseline triglyceride levels of 1.54 to 1.75 mmol/l (about 136 to 154 mg/dl) were treated for 12 weeks with 10 mg/day simvastatin, 9.2 g/day fish oil (Eskimo-3), or a combination of simvastatin and Eskimo-3. The fish oil contained 18% EPA, 12% DHA, and a total of 38% omega-3 fatty acids. Combined treatment significantly reduced triglyceride and LDL-C levels, and significantly increased HDL-C levels, as compared to baseline, and significantly reduced triglyceride levels as compared to simvastatin alone. Liu et al., Nutrition Research 23 (2003) 1027-1034.

An additional study concluded that the combined treatment of low-dose pravastatin and fish oil after dinner in post-renal transplantation dislipidemia is more effective to change the lipid profile after renal transplantation. Grekas et al., Nephron (2001) 88: 329-333. One article summarizes the combination drug therapies for dyslipidemia, including the combination of statins and 3-7 mg fish oil per day. The study indicates that combination therapy may further augment the reduction of triglyceride, total cholesterol, and apoliprotein E levels, as compared with patients on a statin alone. Alaswad et al., Curr. Atheroscler. Rep. (1999) 1:44-49. In another study, it was found that the combination of dietary fish oil and lovastatin reduces both very low-density lipoprotein (VLDL) and low density lipoprotein (LDL). Huff et al., Arterosclerosis and Thrombosis, 12 (8): 901-910 (August 1992).

Additional studies have examined the effects of statins in combination with administration of omega-3 fatty acids and concluded that a diet rich in omega-3 fatty acids increased the cholesterol-lowering effect of simvastatin, counteracted the fasting insulin-elevating effect of simvastatin and did not decrease serum levels of β-carotene and ubiquinol-10. Jula et al., JAMA 287 (5) 598-605 (Feb. 6, 2002). Another study showed an increase in thiobarbituric acid-malondialdehyde complex (TBA-MDA) by using EPA and DHA and statins (e.g., simvastatin) did not affect this result. Grundt et al., Eur. J of Clin. Nutr. (2003) 57: 793-800.

U.S. Pat. No. 6,720,001 discloses a stabilized pharmaceutical oil-in-water emulsion for delivery of a polyfunctional drug having the drug, an aqueous phase, an oil phase and an emulsifier. Statins are claimed among a list of possible polyfunctional drugs, and fish oil is claimed as one of seven optional components for the oil phase. Moreover, U.S. Patent Application Publication No. 2002/0077317 claims compositions of statins and polyunsaturated fatty acids (PUFAs) (EPA and DHA), while U.S. Patent Application Publication No. 2003/0170643 claims a method of treating a patient, by administering a therapeutic which lowers plasma concentrations of apoB and/or an apoB-containing lipoprotein and/or a component of an atherogenic lipoprotein by stimulating post-ER pre-secretory proteolysis (PERPP) using the combination of fish oils with statins, such as pravastatin, lovastatin, simvastatin, atorvastatin, fluvastatin and cerivastatin.

Studies have also investigated the effect of statins and concentrated omega-3 fatty acids, specifically the Omacor® omega-3 acids. For example, Hansen et al. investigated the effect of lovastatin (40 mg/day) in combination with fish oil concentrate (6 g/day Omacor® omega-3 acids) in patients with hypercholesterolemia. Patients having baseline triglyceride levels of 1.66 mmol/l (about 146 mg/dl) were treated with 6 g/day Omacor® for 6 weeks, followed by 40 mg/day lovastatin for an additional 6 weeks, and a combination of both Omacor® and lovastatin for a final 6 weeks. Lovastatin monotherapy resulted in significant increases in HDL-C levels, and significant decreases in triglyceride and LDL-C levels. After combination treatment, triglyceride and LDL-C levels were further significantly decreased. Hansen et al., Arteriosclerosis and Thrombosis 14 (2): 223-229 (February 1994).

Nordoy et al. investigated the effect of atorvastatin and omega-3 fatty acids on patients with hyperlipemia. Patients having baseline triglyceride levels of 3.84 mmol/l (about 337 mg/dl) or 4.22 mmol/l (about 371 mg/dl) were treated with 10 mg/day atorvastatin for 5 weeks. Thereafter, for an additional 5 weeks, atorvastatin treatment was supplemented with 2 g/day Omacor® or placebo. Atorvastatin monotherapy, significantly increased HDL-C levels, and triglyceride and LDL-C levels significantly decreased, as compared to baseline. Combination treatment further increased HDL-C levels, as compared to atorvastatin alone. Triglyceride and LDL-C levels numerically further declined slightly with combination treatment, as compared to atorvastatin monotherapy; however, the decrease was insignificant, and the numerical reduction in triglyceride and LDL-C levels was less than with the reduction experienced by the “atorvastatin+placebo” group. The study concluded that the addition of omega-3 fatty acids to statin (e.g., atorvastatin) treatment was an efficient alternative to treating combined hyperlipemia, as the fatty acids further increased HDL-C and reduced systolic blood pressure, Nordoy et al., Nutr. Metab. Cardiovasc. Dis. (2001) 11:7-16.

Salvi et al. investigated the effects of Omacor® and simvastatin on patients with familial hypercholesterolemia. Patients having baseline triglyceride levels of 1.355 mmol/l (about 119 mg/dl) and already treated with 20-40 mg/day simvastatin were additionally treated with 6 g/day Omacor® for 4 weeks. It was shown that combination treatment significantly decreased triglyceride and LDL-C levels after 2 weeks, as compared to baseline monotherapy. Salvi et al., Curr. Ther. Res. 53:717-21 (1993). Yet another study investigated the effects of omega-3 fatty acids (2 g Omacor® omega-3 acids twice a day) for treating subjects with established CHD and type lib hyperlipidemia who were already taking simvastatin. The study concluded that the Omacor® omega-3 acids was effective in lowering serum triglyceride levels in patients taking simvastatin. Bhatnagar et al., Eur. Heart J Supplements (2001) 4 (Suppl. D): D53-D58.

Chan et al. studied the combined treatment of atorvastatin (40 mg/day) and fish oil (4 Omacor® omega-3 acid capsules orally at night, 4 g/day) on obese, insulin-resistant men with dyslipidemia studied in a fasted state. Patients having baseline triglyceride levels of 1.7 to 2.0 mmol/l (about 150 to 170 mg/dl) were treated for 6 weeks with: 40 mg/day atorvastatin and placebo; 4 g/day Omacor® and placebo; a combination of atorvastatin and Omacor®; or a combination of placebos. Combination treatment significantly decreased triglyceride, non-HDL-C and LDL-C levels, and significantly increased HDL-C, as compared to the placebo group. Chan et al., Diabetes, 51: 2377-2386 (August 2002). An additional paper investigated the effects of atorvastatin (40 mg/day) and fish oil (4 g/day Omacor® omega-3 acids at night) on obese men with dyslipidemia and insulin resistance. The treatment groups received a placebo, atorvastatin, the Omacor® omega-3 acids, or a combination thereof at night. The paper concluded that combination treatment of statins and fish oil may be the optimal approach for correcting dyslipidemia in obese men. Chan et al., Eur. J of Clin. Invest. (2002) 32: 429-436. Another paper investigated the effects of atorvastatin (40 mg/day) and fish oil (4 g/day Omacor® omega-3 acids at night) on plasma high-sensitivity C-reactive protein concentrations in obese individuals with dyslipidemia. The paper concluded that although fish oil supplementation had no effect on plasma hs-CRP, the addition of fish oil to statins may further optimize lipid-regulating effects by enhancing a decrease in plasma triglycerides and increase in HDL-C. Chan et al., Clinical Chemistry (2002) 48 (6): 877-883.

Nordoy et al. investigated the effect of omega-3 fatty acids (3.6 g/day via 4 g/day Omacor® omega-3 acids) and simvastatin (20 mg/day) on patients with combined hyperlipemia. The study concluded that supplementation with the fatty acids reduced hemostatic risk factors and significantly reduced postprandial hyperlipemia. Nordoy et al., Arterioscier. Thromb. Vasc. Biol. (2000) 20:259-265.

Nordoy et al. also investigated the efficiency and the safety of treatment with simvastatin and omega-3 fatty acids in patients with hyperlipidemia. Nordoy et al., J. of Internal Medicine, 243:163-170 (1998). Patients having baseline triglyceride levels of 2.76 mmol/l (about 243 mg/dl) or 3.03 mmol/l (about 266 mg/dl) were treated for 5 weeks with 20 mg/day simvastatin or placebo, then all patients were treated for an additional 5 weeks with 20 mg/day simvastatin. Thereafter, patients were additionally treated with 4 g/day Omacor® or placebo, for a further 5 weeks. The administration of omega-3 fatty acids with simvastatin resulted in moderate reductions in serum total cholesterol and reduction in triglycerol levels. HDL-C levels slightly decreased, and LDL-C levels slightly increased, with the addition of Omacor®, as compared to the baseline monotherapy.

Durrington et al. examined the effectiveness, safety, and tolerability of a combination of Omacor® omega-3 acids and simvastatin in patients with established coronary heart disease and persisting hypertriglyceridemia. Patients having an average baseline triglyceride levels >2.3 mmol/l (average patient serum triglyceride level was 4.6 mmol/l), were treated with 10-40 mg/day simvastatin and 2 g/day Omacor® or placebo, for 24 weeks in a double-blind trial, after which both groups were invited to receive Omacor® for a further 24 weeks in an open study. Combination treatment significantly decreased triglyceride levels within 12 weeks, as compared to baseline monotherapy. In particular, the serum triglyceride levels in patients receiving simvastatin and Omacor® omega-3 acids decreased by 20-30%. In addition, the VLDL cholesterol levels in these patients decreased by 30-40%. LDL-C levels significantly decreased, as compared to baseline monotherapy, only after 48 weeks, although there was a numerical (statistically insignificant) decrease at 12 and 24 weeks. Durrington et al., Heart, 85:544-548 (2001).

U.S. Pat. No. 6,096,338, U.S. Pat. No. 6,267,985, U.S. Pat. No. 6,667,064, U.S. Pat. No. 6,720,001, U.S. Patent Application Publication No. 2003/0082215, U.S. Patent Application Publication No. 2004/0052824, WO 99/29300 and WO 2001/021154 disclose compositions, carrier systems and oil-in-water emulsions containing digestible oils or triglycerides with an active ingredient, such as fenofibrate.

U.S. Pat. No. 6,284,268 is directed to self-emulsifying preconcentrate pharmaceutical compositions capable of forming oil-in-water microemulsions or emulsions upon dilution with an aqueous solution, and containing an omega-3 fatty acid oil and a poorly water soluble therapeutic agent, such as a cyclosporin. The '268 patent formulations use a large amount of surfactant (generally higher than 50% w/w, based on the weight of the solvent system), and less than 10% w/w of a hydrophilic solvent system, to achieve the self-emulsifying compositions. Formulation 19 discloses a self-emulsifying preconcentrate product outside of the scope of the claims of the '268 patent, containing 284 mg of fish oil (about 23% w/w based on the weight of the solvent system, including the fish oil), 663 mg of a surfactant system (about 55% w/w based on the weight of the solvent system), 273 mg of a hydrophilic solvent system (about 22% w/w based on the weight of the solvent system), and 100 mg of fenofibrate. There is no disclosure or suggestion in the '268 patent of a fenofibrate formulation having a solvent system based mainly on fish oil, without the use of a large amount of surfactant. Nor is there any disclosure in the '268 patent regarding administration of the self-emulsifying preconcentrate fenofibrate product to subjects for any treatment. Rather, the '268 patent seemed to use fenofibrate simply to exemplify the solubilizing properties of the self-emulsifying compositions disclosed therein.

Combinations of omega-3 fatty acids with other fibrates, such as gemfibrozil and clofibrate, have not been shown to produce any dramatic synergistic action in the treatment of hyperlipidemia and hyperlipoproteinemia. See Saify et al., Pakistan J. of Pharm. Sci. (2003) 16 (2): 1-8; Pennacchiotti et al., Lipids (2001) 26 (2): 121-127; Wysynski et al., Human and Experimental Toxicology (1993) 12: 337-340.

### SUMMARY OF THE INVENTION

There is an unmet need in the art for combination products of dyslipidemic agents and omega-3 fatty acids, in particular a combination product that provides a single administration of concentrated amounts of omega-3 fatty acids and a dyslipidemic agent, for example, in a unit dosage. There is also an unmet need in the art for a method of administration of a single administration or unit dosage product.

The present invention meets these needs in the art, as well as others, by providing an administration of a unit dosage of a dyslipidemic agent and omega-3 fatty acids that can provide an effective pharmaceutical treatment of coronary heart disease, vascular disease, and related disorders, events, and/or symptoms.

Some embodiments of the present invention provide for a method of utilizing a combination of a dyslipidemic agent and omega-3 fatty acids in the treatment of hypertriglyceridemia, hypercholesterolemia, mixed dyslipidemia, vascular disease, artherosclerotic disease and related conditions, and the prevention or reduction of cardiovascular and vascular events.

In a preferred embodiment, the present invention includes methods of blood lipid therapy in a subject comprising administering to the subject an effective amount of a dyslipidemic agent and an omega-3 fatty acid, wherein the subject has a baseline triglyceride level of 200 to 499 mg/dl and wherein after administration to the subject the triglyceride level and a non-HDL-C level of the subject are reduced without increasing LDL-C as compared to treatment with the dyslipidemic agent alone.

Some embodiments according to the present invention include a method of blood lipid therapy in a subject comprising administering to the subject an effective amount of a dyslipidemic agent and an omega-3 fatty acid, wherein a HDL-C level in the subject is increased and a LDL-C level in the subject is reduced as compared to treatment with the dyslipidemic agent alone.

In further embodiments, the dyslipidemic agent and the omega-3 fatty acid are administered as a single pharmaceutical composition as a combination product, for example, a unit dosage, comprising the dyslipidemic agent and the omega-3 fatty acids.

In preferred embodiments the pharmaceutical compositions comprise Omacor® omega-3 fatty acids, as described in U.S. Pat. Nos. 5,502,077, 5,656,667 and 5,698,594. In other preferred embodiments the pharmaceutical compositions comprise omega-3 fatty acids present in a concentration of at least 40% by weight as compared to the total fatty acid content of the composition.

In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:2 to 2:1.

In variations of the present invention, the dyslipidemic agent is a statin including, but not limited to, simvastatin, rosuvostatin, pravastatin, atorvastatin, lovastatin and fluvastatin. In preferred embodiments the statin used in combination with omega-3 fatty acids is simvastatin.

In one aspect of the invention, the combination product is used in the treatment of hypertriglyceridemia, hypercholesterolemia, mixed dyslipidemia, vascular disease, artherosclerotic disease and related conditions, and the prevention or reduction of cardiovascular and vascular events. Yet other embodiments of the present invention are methods for the treatment of hypertriglyceridemia, the reduction of triglycerides and hypertension comprising a combined administration of a dyslipidemic agent and omega-3 fatty acids.

For example, the methods and compositions of the invention may be used to reduce the LDL-C level of a treated subject. In other embodiments, the triglyceride level of the subject may be reduced. For example, the triglyceride level of the subject may be reduced by at least 10%, preferably about 10% to about 65%, about 15% to about 55%, or about 20% to about 50%, as compared to baseline. In other embodiments, the non-HDL-C level of the subject may be reduced. For example, the non-HDL-C level of the subject may be reduced by at least 10%, preferably about 15% to about 65%, about 25% to about 60% or about 30% to about 55%, as compared to baseline.

In yet further preferred embodiments of the present invention the triglyceride levels in the serum of subjects prior to the first administration to the subject of a combination of a dyslipidemic agent and omega-3 fatty acid is about 200 to about 499 mg/dl.

The invention also includes the use of an effective amount of a dyslipidemic agent and an omega-3 fatty acid for the manufacture of a medicament useful for any of the treatment methods indicated herein.

Other features and advantages of the present invention will become apparent to those skilled in the art upon examination of the following or upon learning by practice of the invention.

### DESCRIPTION OF THE PREFERRED EMBODIMENTS

The present invention is directed to the utilization of dyslipidemic agents and omega-3 fatty acids, preferably concentrated omega-3 fatty acids, for the treatment of hypertriglyceridemia, hypercholesteremia, mixed dyslipidemia, vascular disease, artherosclerotic disease and related conditions and the prevention or reduction of cardiovascular and vascular events and a combination product or unit dosage comprising one or more dyslipidemic agents and one or more omega-3 fatty acids.

In some embodiments, this invention provides a novel combination product for the treatment of hypertriglyceridemia, hypercholesteremia, mixed dyslipidemia, vascular disease, artherosclerotic disease and related conditions, and the prevention or reduction of cardiovascular and vascular events comprising the administration of the combination product to a subject. In a preferred embodiment, the administration comprises omega-3 fatty acids, preferably in the form of the Omacor® omega-3 acids, and a dyslipidemic agent, wherein the omega-3 fatty acids are administered simultaneous to administration of the dyslipidemic agent, e.g., as a single fixed dosage pharmaceutical composition or as separate compositions administered at the same time.

In other preferred embodiments, the administration comprises omega-3 fatty acids and a dyslipidemic agent, wherein the omega-3 fatty acids are administered apart from the administration of the dyslipidemic agent, but in a concomitant treatment regime. For example, the dyslipidemic agent may be administered weekly with daily intake of omega-3 fatty acids. One skilled in the art with the benefit of the present disclosure will understand that the precise dosage and schedule for the administration of the omega-3 fatty acids and the dyslipidemic agent will vary depending on numerous factors, such as, for example, the route of administration and the seriousness of the condition.

In preferred embodiments, the present invention includes methods of blood lipid therapy in a subject comprises administering to the subject an effective amount of a dyslipidemic agent and an omega-3 fatty acid, wherein the subject has a baseline triglyceride level of 200 to 499 mg/dl and wherein after administration to the subject the triglyceride level and a non-HDL-C level of the subject are reduced without increasing LDL-C as compared to treatment with the dyslipidemic agent alone.

In other embodiments, the present invention includes methods of blood lipid therapy in a subject group comprising administering to the subject group an effective amount of a dyslipidemic agent and an omega-3 fatty acid, wherein the subject group has a baseline triglyceride level of 200 to 499 mg/dl and wherein after administration to the subject group the triglyceride level and a non-HDL-C level of the subject group are reduced in a statistically significant amount as compared to a control group treated with the dyslipidemic agent alone without increasing LDL-C in a statistically significant amount as compared to the control group treated with the dyslipidemic agent alone.

Still other embodiments according to the present invention include a method of blood lipid therapy in a subject comprising administering to the subject an effective amount of a dyslipidemic agent and an omega-3 fatty acid, wherein a HDL-C level in the subject is increased and a LDL-C level in the subject is reduced as compared to treatment with the dyslipidemic agent alone. Preferably, the HDL-C level is increased by at least 5%, preferably about 5% to about 30%, preferably by at least 10%, more preferably by at least 15%.

The phrase “compared to treatment with dyslipidemic agent alone” can refer to treatment in the same subject, or treatment of a comparable subject (i.e., a subject within the same class with respect to a particular blood protein, cholesterol or triglyceride level) in a different treatment group.

The present invention may incorporate now known or future known dyslipidemic agents in an amount generally recognized as safe. Preferred dyslipidemic agents include HMG CoA inhibitors including statins, cholesterol absorption inhibitors such as but not limited to ezetimibe, niacin and derivatives such as nicotinamide, CETP inhibitors such as but not limited to torcetrapib, fibrates such as but not limited to fenofibrate, bezafibrate, clofibrate and gemfibrozil, bile acid sequestrants such as but not limited to cholestyramine, cholestipol and colesevelam, MTP inhibitors such as but not limited to those disclosed in WO 00/38725 and Science, 282, 23 Oct. 1998, pp. 751-754, herein incorporated by reference, LXR agonists and/or antagonists, and PPAR agonists and antagonists (such as but not limited to PPAR-alpha, PPAR-gamma, PPAR-delta, PPAR-alpha/gamma, PPAR-gamma/delta, PPAR-alpha/delta, and PPAR-alpha/gamma/delta agonists, antagonists and partial agonists and/or antagonists) such as but not limited to the thiazolidinediones, the non-thiazolidinediones and metaglidasen. There are currently six statins that are widely available: atorvastatin, rosuvastatin, fluvastatin, lovastatin, pravastatin, and simvastatin. A seventh statin, cerivastatin, has been removed from the U.S. market at the time of this writing. However, it is conceivable to one skilled in the art that cerivastatin may be used in conjunction with some embodiments of the present invention if cerivastatin is ultimately determined to be safe and effective.

Generally, the effect of the dyslipidemic agent is dose dependent, i.e., the higher the dose, the greater the therapeutic affect. However, the effect of each dyslipidemic agent is different, and therefore the level of therapeutic effect of one dyslipidemic agent cannot be necessarily be directly correlated to the level of therapeutic effects of other dyslipidemic agents. However, those of ordinary skill in the art would understand the correct dosage to be given to a particular subject, based on experience and the seriousness of the condition.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the Omacor® omega-3 acid (K85EE, Pronova Biocare A.S., Lysaker, Norway) and preferably comprises the following characteristics (per dosage form):

| | Minimum | | |
|---|---|---|---|
| Test | Value | | Maximum Value |
| Eicosapentaenoic acid C20:5 | 430 | mg/g | 495 mg/g |
| Docosahexaenoic acid C22:6 | 347 | mg/g | 403 mg/g |
| EPA and DHA | 800 | mg/g | 880 mg/g |
| Total n-3 fatty acids | 90% | (w/w) | |

The combination product of a dyslipidemic agent and concentrated omega-3 fatty acids may be administered in a capsule, a tablet, a powder that can be dispersed in a beverage, or another solid oral dosage form, a liquid, a soft gel capsule or other convenient dosage form such as oral liquid in a capsule, as known in the art. In some embodiments, the capsule comprises a hard gelatin. The combination product may also be contained in a liquid suitable for injection or infusion.

The active ingredients of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include coilloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

Excipients include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The combination product of a dyslipidemic agent and concentrated omega-3 fatty acids is aided by the solubility of the dyslipidemic agent in the omega-3 fatty acid oil. Thus, the combination product does not require high amounts of solubilizers, such as surfactants, cosolvents, oils or combinations thereof. Preferably, the active ingredients are administered without the use of large amounts of solubilizers (other than the omega-3 fatty acid oil). In preferred embodiments, if present at all, solubilizers other than the omega-3 fatty acid oil are present in amounts of less than 50% w/w based on the total weight of the solvent system in the dosage form(s), preferably less than 40%, more preferably less than 30%, even more preferably less than 20%, still more preferably less than 10% and most preferably less than 5%. In some embodiments, the solvent system contains no solubilizers other than the omega-3 fatty acid oil. As used herein, “solvent system” includes the omega-3 fatty acid oil. In other preferred embodiments, the weight ratio of omega-3 fatty acid oil to other solubilizer is at least 0.5 to 1, more preferably at least 1 to 1, even more preferably at least 5 to 1, and most preferably at least 10 to 1.

In other preferred embodiments, if present at all, the amount of hydrophilic solvent used in the solvent system is less than 20% w/w based on the total weight of the solvent system in the dosage form(s), more preferably less than 10%, and most preferably less than 5%. In certain embodiments, the amount of hydrophilic solvent used in the solvent system is between 1 and 10% w/w.

In preferred embodiments, omega-3 fatty acid oil is present in amounts of at least 30% w/w based on the total weight of the solvent system in the dosage form(s), more preferably at least 40%, even more preferably at least 50%, and most preferably at least 60%. In certain embodiments, the amount can be at least 70%, at least 80% or at least 90%.

The dosage form is stable at room temperature (about 23° C. to 27° C.) for a period of at least one month, preferably at least six months, more preferably at least one year, and most preferably at least two years. By “stable”, applicants mean that the solubilized dyslipidemic agent does not come out of solution to any appreciable degree, for example, in amounts of less than 10%, preferably less than 5%.

The concentrated omega-3 fatty acids can be administered in a daily amount of from about 0.1 g to about 10 g, more preferably about 1 g to about 6 g, and most preferably from about 2 g to about 4 g.

The dyslipidemic agent may be administered in an amount more than, equal to or less than the conventional full-strength dose as a single-administered product. For example, the dyslipidemic agent may be administered in an amount of from 10-100%, preferably about 25-100%, most preferably about 50-80%, of the conventional full-strength dose as a single-administered product. In one embodiment of the present invention, the statin can generally be present in an amount from about 0.5 mg to 80 mg, more preferably from about 1 mg to about 40 mg, and most preferably from about 5 mg to about 20 mg, per gram of omega-3 fatty acid. The daily dose may range from about 2 mg to about 320 mg, preferably about 4 mg to about 160 mg.

In some variations of the present invention, the combination of dyslipidemic agent and the omega-3 fatty acids is formulated into a single administration or unit dosage. In preferred embodiments, a statin is utilized selected from the following group: atorvastatin, rosuvastatin, fluvastatin, lovastatin, pravastatin, and simvastatin.

Pravastatin, which is known in the market as Pravachol® manufactured by Bristol-Myers Squibb, Princeton, N.J., is hydrophilic. Pravastatin is best absorbed without food, i.e., an empty stomach. The dosage of pravastatin, in the combined administration of concentrated omega-3 fatty acids is preferably from 2.5 to 80 mg, preferably 5 to 60, and more preferably from 10 to 40 mg per dosage of concentrated omega-3 fatty acids. In one variation, the combination product using pravastatin is taken at or around bedtime, e.g., 10 pm.

Lovastatin, which is marketed under the name Mevacor® by Merck, Whitehouse Station, N.J., is hydrophobic. Unlike pravastatin, lovastatin should be taken with meals and accordingly, in some embodiments, the combination product of concentrated omega-3 fatty acids and lovastatin should be taken with food. The dosage of lovastatin, in the combined administration of concentrated omega-3 fatty acids is preferably from 2.5 to 100 mg, preferably 5 to 80 mg, and more preferably from 10 to 40 mg per dosage of concentrated omega-3 fatty acids.

Simvastatin, which is marketed under the name Zocor® by Merck, Whitehouse Station, N.J., is hydrophobic. The dosage of simvastatin, in the combined administration of concentrated omega-3 fatty acids is preferably from 1 to 80 mg per day, preferably 2 to 60 mg, and more preferably from 5 to 40 mg per dosage of concentrated omega-3 fatty acids.

Atorvastatin, which is marketed under the name Lipitor® by Pfizer, New York, N.Y., is hydrophobic and is known as a synthetic statin. The dosage of atorvastatin, in the combined administration of concentrated omega-3 fatty acids is preferably from 2.5 to 100 mg, preferably 5 to 80 mg, and more preferably from 10 to 40 mg per dosage of concentrated omega-3 fatty acids.

Fluvastatin, which is marketed under the name Lescol® by Novartis, New York, N.Y., is hydrophilic and is known as a synthetic statin. The dosage of fluvastatin, in the combined administration of concentrated omega-3 fatty acids is from 5 to 160 mg, preferably 10 to 120 mg, and more preferably from 20 to 80 mg per dosage of concentrated omega-3 fatty acids.

Rosuvastatin is marketed under the name Crestor® by Astra Zeneca, Wilmington, Del. The dosage of rosuvastatin, in the combined administration of concentrated omega-3 fatty acids is from 1 to 80 mg, preferably 2 to 60 mg, and more preferably from 5 to 40 mg per dosage of concentrated omega-3 fatty acids.

The daily dosages of dyslipidemic agent and concentrated omega-3 fatty acids can be administered together in from 1 to 10 dosages, with the preferred number of dosages from 1 to 4 times a day, most preferred 1 to 2 times a day. The administration is preferably oral administration, although other forms of administration that provides a unit dosage of dyslipidemic agent and concentrated omega-3 fatty acids may be used.

In some embodiments, the formulations of the present invention allow for improved effectiveness of each active ingredient, with one or both administered as a conventional full-strength dose, as compared to the formulations in the prior art. In other embodiments, the formulations of the present invention may allow for reduced dosages of dyslipidemic agent and/or omega-3 fatty acids, as compared to the formulations in the prior art, while still maintaining or even improving upon the effectiveness of each active ingredient.

The present combination of a dyslipidemic agent and concentrated omega-3 fatty acids may allow for a greater effect than any expected combined or additive effect of the two drugs alone. Moreover, the combined or additive effect of the two drugs may depend on the initial level of triglycerides in the blood of a subject. For example, the triglyceride level of a subject is generally as normal if less than 150 mg/dL, borderline to high if within about 150-199 mg/dL, high if within about 200-499 mg/dL and very high if 500 mg/dL or higher. The present invention may be used to reduce the triglyceride level of a “very high” down to a “high” or “borderline to high” in less than 48 weeks, preferably within 24 weeks, more preferably within 12 weeks, and most preferably within 6 weeks, 4 weeks or 2 weeks. The present invention may also be used to reduce the triglyceride level of a “high” down to a “borderline to high” or “normal” in less than 48 weeks, preferably within 24 weeks, more preferably within 12 weeks, and most preferably within 6 weeks, 4 weeks or 2 weeks.

Thus, the combined treatment of the two active ingredients, separately or through the novel combination product of the present invention, may cause an unexpected increase in effect of the active ingredients that allows increased effectiveness with standard dosages or maintained effectiveness with reduced dosages of the two active ingredients. It is well accepted in practice that an improved bioavailability or effectiveness of a drug or other active ingredient allows for an appropriate reduction in the daily dosage amount. Any undesirable side effects may also be reduced as a result of the lower dosage amount and the reduction in excipients (e.g., surfactants).

The utilization of a single administration of a combination of a dyslipidemic agent and concentrated omega-3 fatty acids overcomes the limitations of the prior art by improving the efficacy of the dyslipidemic agent and the concentrated omega-3 fatty acids, and allows for a treatment with improved effectiveness and less excipients than in multiple administrations of omega-3 fatty acids and dyslipidemic agents.

The administration of a combination of dyslipidemic agent and concentrated omega-3 fatty acids achieves results that are highly advantageous and beneficial to the pharmaceutical and medicinal arts. The increased efficacy of the combined treatment and combination product allows for a novel and more efficient pharmaceutical treatment for hypertriglyceridemia, hypercholesterolemia, mixed dyslipidemia, vascular disease, artherosclerotic disease and related conditions, the prevention or reduction of cardiovascular and vascular events.

### EXAMPLES

The effect of 4 grams per day of Omacor® omega-3 fatty acids on the lipid parameters, i.e. triglyceride levels (TG), total cholesterol, high density lipoproteins (HDL), low density lipoproteins (LDL) and very low density lipoprotein (VLDL), of patients with different baseline TG levels has been evaluated. The Omacor® omega-3 fatty acids were supplied as a liquid-filled gel capsule for oral administration. Each one gram capsule of Omacor® contained at least 900 mg of ethyl esters of omega-3 fatty acids, which comprises predominantly eicosapentaenoic acid (EPA) (about 465 mg) and docosahexaenoic acid (DHA) (about 375 mg). As shown in Table 1, the effectiveness of Omacor® omega-3 fatty acids is dependent on the baseline TG levels of the treated of patients.

TABLE 1
Percent Change in Lipid Parameters in Patients after
administration of Omacor ® as Monotherapy

| Baseline TG | | Total | | | | |
|---|---|---|---|---|---|---|
| (mg/dL) | TG | cholesterol | HDL | LDL | VLDL | Non-HDL |
| 0-199 | −22.5 | 3.5 | 5.2 | 10.7 | −31.6 | 3.8 |
| 200-299 | −23.0 | 0.2 | 7.3 | 5.9 | −21.2 | −0.5 |
| 300-399 | −26.1 | −1.1 | 6.1 | 9.9 | −22.3 | −1.2 |
| 400-499 | −25.9 | −4.7 | 12.6 | 18.9 | −8.8 | −7.3 |
| 500-599 | −39.8 | −4.8 | 9.8 | 44.7 | −34.9 | −6.2 |
| 600-699 | −36.9 | −3.6 | 8.1 | 47.6 | −25.6 | −5.0 |
| 700- | −39.9 | −15.4 | 16.5 | 40.3 | −26.0 | −17.8 |

The effects of Omacor® omega-3 fatty acids co-administered with simvastatin was evaluated in a study of 20 patients. The patients were initially treated with 40 mg of simvastatin to establish baseline triglyceride levels between 200 and 499 mg/dL. After baseline triglyceride levels were established, the patients were treated with a combination of 4 grams per day of Omacor® omega-3 fatty acids and 40 mg of simvastatin over an 8 week period.

As shown in Tables 2 and 3, the administration of a combination of Omacor® omega-3 fatty acids and simvastatin reduced triglyceride, total cholesterol, non-HDL cholesterol and LDL cholesterol in the serum of treated patients relative to baseline (simvastatin treatment alone). In addition, the administration of a combination of Omacor® omega-3 fatty acids and simvastatin increased the levels of HDL cholesterol in the treated patients relative to placebo. Surprisingly, as compared to Omacor® treatment alone, non-HDL cholesterol levels were reduced without an increase in LDL cholesterol levels.

TABLE 2
Median Baseline and Percent Change from Baseline in Lipid
Parameters in Patients with TG Levels 200-499 mg/dL
After 4 Weeks

| | | % Change | |
|---|---|---|---|
| | Baseline (after | After 4 Wks | |
| | Administration of | Administration of | |
| | Simvastatin | Simvastatin | |
| | 40 mg/day) | 40 mg/day and | p-value vs. |
| | (mg/dL) | Omacor ® 4 g/day. | Baseline |
| Total Cholesterol | 186.7 | −13.4 | 0.0050 |
| Non-HDL-C | 146.7 | −20.6 | <0.0001 |
| HDL-C | 40.0 | +13.4 | 0.0879 |
| LDL-C | 101.0 | −8.7 | 0.0149 |
| TG | 256.7 | −36.9 | 0.0020 |

TABLE 3
Median Baseline and Percent Change from Baseline in Lipid
Parameters in Patients with TG Levels 200-499 mg/dL
After 8 Weeks

| | | % Change | |
|---|---|---|---|
| | Baseline (after | After 8 Wks | |
| | Administration of | Administration of | |
| | Simvastatin | Simvastatin | |
| | 40 mg/day) | 40 mg/day and | p-value vs. |
| | (mg/dL) | Omacor ® 4 g/day. | Baseline |
| Total Cholesterol | 186.7 | −15 | <0.0001 |
| Non-HDL-C | 146.7 | −22 | <0.0001 |
| HDL-C | 40.0 | +8 | 0.0846 |
| LDL-C | 101.0 | −4 | 0.257 |
| TG | 256.7 | −23 | 0.002 |

The following formulations may be prepared in accordance with the invention:

Formulation 1:

| Ingredient | Mg/capsule |
|---|---|
| K85EE | 1000 |
| Dehydrated ethanol | 39.5 |
| Capmul ® MCM | 20 |
| Simvastatin | 20 |

Formulation 2:

| Ingredient | Mg/capsule |
|---|---|
| K80EE | 1000 |
| Dehydrated ethanol | 50 |
| Propylene glycol | 20 |
| monocaprylate | |
| Ezetimibe | 5 |

Formulation 3:

| Ingredient | Mg/capsule |
|---|---|
| K85EE | 1000 |
| Glycerol | 35 |
| Polyethoxylated castor | 25 |
| oil | |
| Pioglitazone | 15 |

Formulation 4:

| Ingredient | Mg/capsule |
|---|---|
| EPAX7010EE | 1000 |
| Propylene glycol | 30 |
| Olive oil | 50 |
| Atorvastatin | 10 |

1. A method of blood lipid therapy in a subject comprising administering to the subject a pharmaceutical composition comprising an effective amount of a fibrate and natural or synthetic omega-3 fatty acids or pharmaceutically acceptable esters, derivatives, conjugates, precursors or salts thereof, or mixtures thereof, wherein the subject has a baseline triglyceride level of 200 to 499 mg/dl and wherein after administration to the subject the triglyceride level and a non-HDL-C level of the subject are reduced without increasing LDL-C as compared to treatment with the fibrate alone.

2-3. (canceled)

4. The method of claim 1, wherein the omega-3 fatty acids are present in a concentration of at least 40% by weight as compared to the total fatty acid content of the composition.

5. The method of claim 1, wherein the omega-3 fatty acids are present in a concentration of at least 80% by weight as compared to the total fatty acid content of the composition.

6. The method of claim 1, wherein the omega-3 fatty acids comprise at least 50% by weight of EPA and DHA as compared to the total fatty acid content of the composition.

7. The method of claim 1, wherein the omega-3 fatty acids comprise at least 80% by weight of EPA and DHA as compared to the total fatty acid content of the composition.

8. The method of claim 1, wherein the omega-3 fatty acids comprise about 5% to about 95% by weight of EPA as compared to the total fatty acid content of the composition.

9. The method of claim 1, wherein the omega-3 fatty acids comprise about 40% to about 55% by weight of EPA as compared to the total fatty acid content of the composition.

10. The method of claim 1, wherein the omega-3 fatty acids comprise about 5% to about 95% by weight of DHA as compared to the total fatty acid content of the composition.

11. The method of claim 1, wherein the omega-3 fatty acids comprise about 30% to about 60% by weight of DHA as compared to the total fatty acid content of the composition.

12. The method of claim 1, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

13. The method of claim 1, wherein the omega-3 fatty acids comprise EPA and DHA in a ratio of EPA:DHA from 99:1 to 1:99.

14. The method of claim 1, wherein the omega-3 fatty acids comprise EPA and DHA in a ratio of EPA:DHA from 2:1 to 1:2.

15. The method of claim 1, wherein the omega-3 fatty acids are administered apart from administration of the fibrate.

16. The method of claim 1, wherein the omega-3 fatty acids and the fibrate are administered together in a unit dose form.

17. (canceled)

18. The method of claim 1, wherein a LDL-C level of the subject is reduced.

19. The method of claim 1, wherein the triglyceride level of the subject is reduced by about 10% to about 65%, as compared to baseline.

20. The method of claim 1, wherein the non-HDL-C level of the subject is reduced by about 10% to about 55%, as compared to baseline.

21. The method of claim 1, wherein the triglyceride level and the non-HDL-C level in the subject are reduced before 48 weeks of therapy.

22. A method of blood lipid therapy in a subject comprising administering to the subject a pharmaceutical composition comprising an effective amount of a fibrate and natural or synthetic omega-3 fatty acids or pharmaceutically acceptable esters, derivatives, conjugates, precursors or salts thereof, or mixtures thereof, wherein a HDL-C level in the subject is increased and a LDL-C level in the subject is reduced as compared to treatment with the fibrate alone.

23-24. (canceled)

25. The method of claim 22, wherein the subject has a baseline triglyceride level of 200 to 499 mg/dl.

26. The method of claim 22, wherein the HDL-C level of the subject is increased by about 5% to about 25%.

27-32. (canceled)

33. A method of blood lipid therapy in a subject group comprising administering to the subject group a pharmaceutical composition comprising an effective amount of a fibrate and natural or synthetic omega-3 fatty acids or pharmaceutically acceptable esters, derivatives, conjugates, precursors or salts thereof, or mixtures thereof, wherein the subject group has a baseline triglyceride level of 200 to 499 mg/dl and wherein after administration to the subject group the triglyceride level and a non-HDL-C level of the subject group are reduced in a statistically significant amount as compared to a control group treated with the fibrate alone without increasing LDL-C in a statistically significant amount as compared to the control group treated with the fibrate alone.
